(19) United States
(12) Patent Application Publication
(10) Pub. No.: US 2024/0342136 A1
(43) Pub. Date: Oct. 17, 2024

(54) Glutamate receptor agonists for suppression of mast cell function

(71) Applicant: University of Pittsburgh - Of the Commonwealth System of Higher Education, Pittsburgh, PA (US)

(72) Inventor: Daniel H. Kaplan, Pittsburgh, PA (US)

(21) Appl. No.: 18/631,595

(22) Filed: Apr. 10, 2024

Related U.S. Application Data
(60) Provisional application No. 63/458,450, filed on Apr. 11, 2023.

(51) Int. Cl. A61K 31/40; A61K 9/00; A61K 31/194; A61P 29/00; A61P 37/08

(52) U.S. Cl. CPC A61K 31/40; A61K 9/0014; A61K 31/194; A61P 29/00; A61P 37/08

(57) ABSTRACT

Provided herein is a method of treating a disease associated with activation of mast cells in a patient. The method comprises administering to the patient an amount of a GluR6 glutamate receptor agonist effective to reduce responsiveness of a mast cell in the patient. Pharmaceutical compositions comprising a GluR6 glutamate receptor agonist also are provided.

### CROSS REFERENCE TO RELATED APPLICATIONS

This application claims priority to U.S. Provisional Patent Application No. 63/458,450 filed Apr. 11, 2023, entitled “Glutamate Receptor Agonists to Suppress Mast Cell Function”, the disclosure of which is incorporated herein by reference in its entirety.

### STATEMENT REGARDING FEDERAL FUNDING

This invention was made with government support under AR077341 awarded by the National Institutes of Health. The government has certain rights in the invention.

Mast cell activation plays a role in many diseases and as knowledge of disease etiologies grow, there are a growing number of diseases involving mast cell activation, such as degranulation. Mast cells are myeloid-derived immune cells. They arise in the bone marrow, and then circulate and home to various tissues. Under the influence of stem cell factors produced locally in the tissue, the progenitor cells then differentiate into mature mast cells, influenced by locally-produced factors. Mature mast cells are present only in tissue and typically do not circulate. Mast cells are commonly found in connective tissue in virtually every organ. Both exogenous and endogenous stimuli can induce the mast cells to degranulate, thereby releasing inflammatory mediators and facilitating the induction, amplification, and persistence of inflammatory cascades.

Examples of diseases associated with mast cell activation include: type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barré syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, or a disease resulting from, or caused by, cutaneous mast cell activation (see, e.g., da Silva E Z, et al. Mast cell function: a new vision of an old cell. J Histochem Cytochem. 2014 October; 62(10):698-738). In the gut, mast cell activation is associated with inflammatory bowel disease (IBD) such as Crohn's disease and ulcerative colitis, and irritable bowel syndrome (IBS). In the airway, mast cell activation is associated with asthma, anaphylaxis, eosinophilic esophagitis, and allergic rhinitis. Mast cell activation is also involved in food allergies (see, e.g., Leru P M. Evaluation and Classification of Mast Cell Disorders: A Difficult to Manage Pathology in Clinical Practice. Cureus. 2022 Feb. 13; 14(2):e22177).

Treatments for diseases that involve mast cell activation are desired.

### SUMMARY

A method of treating a disease associated with activation of mast cells in a patient is provided. The method comprises administering to the patient an amount of a GluR6 glutamate receptor agonist effective to reduce responsiveness of a mast cell in the patient.

A method of suppressing activation of mast cells is provided. The method comprises contacting the mast cell with a GluR6 glutamate receptor agonist, in an amount effective to reduce responsiveness of the mast cell.

A topical drug composition is provided. The composition comprises GluR6 glutamate receptor agonist and a pharmaceutically-acceptable excipient.

The following numbered clauses describe various aspects, embodiments, and/or examples of the present invention.

Clause 1. A method of treating a disease associated with activation of mast cells in a patient, comprising administering to the patient an amount of a GluR6 glutamate receptor agonist effective to reduce responsiveness of a mast cell in the patient.

Clause 2. The method of clause 1, comprising inducing hyporesponsiveness in the mast cells.

Clause 3. The method of clause 1 or 2, wherein the mast cells are cutaneous mast cells.

Clause 4. The method of any one of clauses 1-3, wherein the patient is a vertebrate.

Clause 5. The method of clause 4, wherein the patient is human.

Clause 6. The method of any one of clauses 1-5, wherein the disease is one or more of type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barré syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, a disease resulting from, or caused by, cutaneous mast cell activation, inflammatory bowel disease (IBD) such as Crohn's disease and ulcerative colitis, irritable bowel syndrome (IBS), asthma, anaphylaxis, eosinophilic esophagitis, allergic rhinitis, and food allergies.

Clause 7. The method of any one of clauses 1-5, wherein the disease is one of atopic dermatitis, psoriasis, rosacea, eczema, contact dermatitis, mastocytosis, skin inflammation, urticaria, urticarial diseases in a patient.

Clause 8. The method of any one of clauses 1-5, wherein the disease is atopic dermatitis.

Clause 9. The method of any one of clauses 1-5, wherein the disease is rosacea.

Clause 10. The method of any one of clauses 1-5, wherein the disease is urticaria or urticarial disease.

Clause 11. The method of any one of clauses 1-5, wherein the disease is mastocytosis.

Clause 12. The method of any one of clauses 1-11, wherein the GluR6 glutamate receptor agonist is administered topically to the patient.

Clause 13. The method of any one of clauses 1-12, wherein the GluR6 glutamate receptor agonist comprises 2-Amino-4-methylpentanedioic acid, or a pharmaceutically-acceptable salt thereof.

Clause 14. The method of any one of clauses 1-12, wherein the GluR6 glutamate receptor agonist comprises (2S,4R)-2-Amino-4-methylpentanedioic acid, or a pharmaceutically acceptable salt thereof.

Clause 15. The method of any one of clauses 1-12, wherein the GluR6 glutamate receptor agonist comprises glutamate, kainic acid, or domoic acid.

Clause 16. The method of any one of clauses 1-12, wherein the GluR6 glutamate receptor agonist comprises a compound having the structure of Formula (I), as follows:

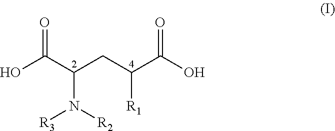

wherein:
   R₁ is C1-C3 alkyl (e.g. methyl, ethyl, n-propyl, isopropyl, or cyclopropyl), halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
   R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3,
or a pharmaceutically acceptable salt thereof.

Clause 17. The method of clause 16, wherein R₁ is methyl, and one of R₂ and R₃ is H and the other of R₂ and R₃ is H, methyl, —CHO, or COCH₃.

Clause 18. The method of any one of clauses 1-17, wherein the GluR6 glutamate receptor agonist is administered to a concentration ranging from 100 pM (picomolar) to 10 mM (millimolar).

Clause 19. A method of suppressing activation of mast cells, comprising contacting the mast cell with a GluR6 glutamate receptor agonist, in an amount effective to reduce responsiveness of the mast cell.

Clause 20. The method of clause 19, comprising inducing hyporesponsiveness in the mast cells.

Clause 21. The method of clause 19 or 20, wherein the mast cells are cutaneous mast cells.

Clause 22. The method of any one of clauses 19-21, for treatment of a disease associated with mast cell activation in a patient, comprising administering to the patient an amount of a GluR6 glutamate receptor agonist effective to reduce responsiveness of a mast cell in the patient.

Clause 23. The method of clause 22, wherein the patient is a vertebrate.

Clause 24. The method of clause 22, wherein the patient is human.

Clause 25. The method of any one of clauses 22-24, wherein the disease is one or more of type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barré syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, a disease resulting from, or caused by, cutaneous mast cell activation, inflammatory bowel disease (IBD) such as Crohn's disease and ulcerative colitis, irritable bowel syndrome (IBS), asthma, anaphylaxis, eosinophilic esophagitis, allergic rhinitis, and food allergies.

Clause 26. The method of any one of clauses 22-24, wherein the disease is one of atopic dermatitis, psoriasis, rosacea, eczema, contact dermatitis, mastocytosis, skin inflammation urticaria, urticarial diseases in a patient.

Clause 27. The method of any one of clauses 22-24, wherein the disease is atopic dermatitis.

Clause 28. The method of any one of clauses 22-24, wherein the disease is rosacea.

Clause 29. The method of any one of clauses 22-24, wherein the disease is urticaria or urticarial disease.

Clause 30. The method of any one of clauses 22-24, wherein the disease is mastocytosis.

Clause 31. The method of any one of clauses 22-30, wherein the GluR6 glutamate receptor agonist is administered topically to the patient.

Clause 32. The method of any one of clauses 19-31, wherein the GluR6 glutamate receptor agonist comprises 2-Amino-4-methylpentanedioic acid, or a pharmaceutically-acceptable salt thereof.

Clause 33. The method of any one of clauses 19-31, wherein the GluR6 glutamate receptor agonist comprises (2S,4R)-2-Amino-4-methylpentanedioic acid, or a pharmaceutically acceptable salt thereof.

Clause 34. The method of any one of clauses 19-31, wherein the GluR6 glutamate receptor agonist comprises glutamate, kainic acid, or domoic acid.

Clause 35. The method of any one of clauses 19-31, wherein the GluR6 glutamate receptor agonist comprises a compound having the structure of Formula (I), as follows:

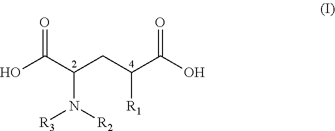

wherein:
   R₁ is C1-C3 alkyl (e.g. methyl, ethyl, n-propyl, isopropyl, or cyclopropyl), halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
   R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3,
or a pharmaceutically acceptable salt thereof.

Clause 36. The method of clause 35, wherein R₁ is methyl, and one of R₂ and R₃ is H and the other of R₂ and R₃ is H, methyl, —CHO, or COCH₃.

Clause 37. The method of any one of clauses 19-36, wherein the GluR6 glutamate receptor agonist is contacted with the mast cells at a concentration ranging from 100 pM (picomolar) to 10 mM (millimolar).

Clause 38. A topical drug composition, comprising GluR6 glutamate receptor agonist and a pharmaceutically-acceptable excipient.

Clause 39. The composition of clause 38 comprising: a compound having the structure of Formula (I), as follows:

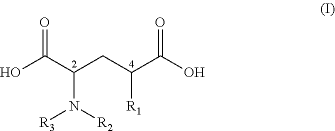

wherein:
   R₁ is C1-C3 alkyl (e.g. methyl, ethyl, n-propyl, isopropyl, or cyclopropyl), halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
   R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3,
or a pharmaceutically acceptable salt thereof,
in a concentration (e.g., % wt., or any useful measure of concentration or amount of an active ingredient in a composition) for delivery of an amount of the GluR6 glutamate receptor agonist effective to treat a disease associated with activation of mast cells in a patient.

Clause 40. The composition of clause 39, comprising 2-Amino-4-methylpentanedioic acid, or a pharmaceutically-acceptable salt thereof.

Clause 41. The composition of clause 39, comprising 2S,4R)-2-Amino-4-methylpentanedioic acid, or a pharmaceutically acceptable salt thereof.

### BRIEF DESCRIPTION OF THE DRAWINGS

FIGS. 1A-1F provide in vitro data supporting suppression of mast cell degranulation by SYM2081 agonism of GluK2.

FIGS. 2A-2D provide in vivo data to support suppression of mast cell activation with SYM2081 agonism of GluK2.

FIGS. 3A and 3B provide in vivo data to support suppression of mast cell activation with topical SYM2081.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

As used herein, the “treatment” or “treating” of a patient means administration to a patient by any suitable dosage regimen, procedure and/or administration route of a composition, device, or structure with the object of achieving a desirable clinical/medical end-point, including but not limited to, inducing mast cell hyporesponsiveness or treatment of a disease associated with mast cell activation. The therapeutic agent may be administered by any effective route, but in the context of inducing mast cell hyporesponsiveness or treatment of a disease associated with mast cell activation, may be most typically delivered parenterally, e.g., as an intravenous or infusion therapy, orally, or topically to a patient. The therapeutic agent may be administered as a single dose, continuously, at regular or irregular intervals, or in amounts and intervals as dictated by any clinical parameter of a patient.

As used herein, “alkyl” refers to straight, branched chain, or cyclic hydrocarbon groups including, for example, from 1 to about 20 carbon atoms, for example and without limitation C1-C3, C1-C6, C1-C10 groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C1, C2, C3, C4, C5, C6, C7, C8, C9, or C10 group that is substituted or unsubstituted. “lower alkyl” refers to C1-C6 alkyl. Non-limiting examples of straight alkyl groups include methyl, ethyl, propyl, butyl, pentyl, hexyl, heptyl, octyl, nonyl, and decyl. Branched alkyl groups comprise any straight alkyl group substituted with any number of alkyl groups. Non-limiting examples of branched alkyl groups include isopropyl, n-butyl, isobutyl, sec-butyl, and t-butyl. “Unsaturated alkyl” may comprise one or more, e.g., 1, 2, 3, 4, or 5, carbon-to-carbon double bonds and alternatively may be referred to as alkene or alkenyl, as described below. “Substituted alkyl” can include alkyl substituted at 1 or more (e.g., 1, 2, 3, 4, 5, 6, or more) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” can include alkylene or substituted alkylene.

Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. A cycloalkyl group may be attached via any atom. Cycloalkyl also contemplates fused rings where the cycloalkyl is fused to an aryl or hetroaryl ring. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with at least 1, 2 or 3 substituents, attached at any available atom to produce a stable compound, wherein the substituents are as described herein. A cycloalkylene may be formed by two “R groups” taken together, such as with “R₂ and R₃ taken together,” as referenced below.

“Alkene or alkenyl” can include straight, branched chain, or cyclic hydrocarbyl groups including, e.g., from 2 to about 20 carbon atoms having one or more, e.g., 1, 2, 3, 4, or 5, carbon-to-carbon double bonds, and may be referred to as “unsaturated alkyl”. The olefin or olefins of an alkenyl group can be, for example, E, Z, cis, trans, terminal, or exo-methylene. An alkenyl or alkenylene group can be, for example, a C2, C3, C4, C5, C6, C7, C8, C9, C10, C11, C12, C13, C14, C15, C16, C17, C18, C19, or C20 group that is substituted or unsubstituted. A halo-alkenyl group can be any alkenyl group substituted with any number of halogen atoms. “Substituted alkene” can include alkene substituted at 1 or more, e.g., 1, 2, 3, 4, or 5 positions, which substituents are attached at any available atom to produce a stable compound, with substitution as described herein. “Optionally substituted alkene” can include alkene or substituted alkene. Likewise, “alkenylene” can refer to divalent alkene. Examples of alkenylene include without limitation, ethenylene (—CH═CH—) and all stereoisomeric and conformational isomeric forms thereof. “Substituted alkenylene” can refer to divalent substituted alkene. “Optionally substituted alkenylene” can refer to alkenylene or substituted alkenylene.

Alkyne or “alkynyl” refers to a straight, branched chain, or cyclic unsaturated hydrocarbon having the indicated number of carbon atoms and at least one triple bond. The triple bond of an alkyne or alkynyl group can be internal or terminal. Examples of a (C2-C8)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below. An alkyne or alkynyl group can be, for example, a C2, C3, C4, C5, C6, C7, C8, C9, C10, C11, C12, C13, C14, C15, C16, C17, C18, C19, or C20 group that is substituted or unsubstituted. A halo-alkynyl group can be any alkynyl group substituted with any number of halogen atoms. The term “alkynylene” refers to divalent alkyne. Examples of alkynylene include without limitation, ethynylene, propynylene. “Substituted alkynylene” refers to divalent substituted alkyne.

“Carboxyl” or “carboxylic” refers to group having an indicated number of carbon atoms, where indicated, and terminating in a —C(O)OH group, thus having the structure —R—C(O)OH, where R is an unsubstituted or substituted divalent organic group that can include linear, branched, or cyclic hydrocarbons. Non-limiting examples of these include: C1-C8 carboxylic groups, such as ethanoic, propanoic, 2-methylpropanoic, butanoic, 2,2-dimethylpropanoic, pentanoic, etc. “Amine” or “amino” refers to group having the indicated number of carbon atoms, where indicated, and terminating in a —NH₂ group, thus having the structure —R—NH₂, where R is an unsubstituted or substituted divalent organic group that, e.g., includes linear, branched, or cyclic hydrocarbons, and optionally comprises one or more heteroatoms. The term “alkylamino” refers to a radical of the formula —NHRₓ or —NRₓRₓ where each Rₓ is, independently, an alkyl radical as defined above. “Alkoxyl” or “alkoxy” refers to an —O-alkyl group.

“Aryl,” alone or in combination refers to an aromatic ring system such as phenyl or naphthyl. “Aryl” also can include aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” refers to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy. “Arylene” denotes divalent aryl, and “substituted arylene” refers to divalent substituted aryl. “Optionally substituted arylene” refers to arylene or substituted arylene. A “polycyclic aryl group” and related terms, such as “polycyclic aromatic group” refers to a group composed of at least two fused aromatic rings. “Heteroaryl” or “hetero-substituted aryl” refers to an aryl group substituted with one or more heteroatoms, such as N, O, P, and/or S. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

Terms not referenced above may take on a meaning as is understood to those of ordinary skill in the chemical and pharmaceutical arts.

Terms combining the foregoing refer to any suitable combination of the foregoing, such as arylalkenyl, arylalkynyl, heteroarylalkyl, heteroarylalkenyl, heteroarylalkynyl, heterocyclylalkyl, heterocyclylalkenyl, heterocyclylalkynyl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, and alkynylhereroaryl. As an example, “arylalkylene” refers to a divalent alkylene wherein one or more hydrogen atoms in an alkylene group is replaced by an aryl group, such as a (C3-C8)aryl group. Examples of (C3-C8)aryl-(C1-C6)alkylene groups include without limitation 1-phenylbutylene, phenyl-2-butylene, I-phenyl-2-methylpropylene, phenylmethylene, phenylpropylene, and naphthylethylene. The term “(C3-C8)cycloalkyl-(C1-C6)alkylene” refers to a divalent alkylene wherein one or more hydrogen atoms in the C1-C6 alkylene group is replaced by a (C3-C8)cycloalkyl group. Examples of (C3-C8)cycloalkyl-(C1-C6)alkylene groups include without limitation 1-cycloproylbutylene, cycloproyl-2-butylene, cyclopentyl-1-phenyl-2-methylpropylene, cyclobutylmethylene, and cyclohexylpropylene.

Diseases associated with mast cell activation, including mast cell mediator disorders, include, without limitation: type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barré syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, a disease resulting from, or caused by, cutaneous mast cell activation, inflammatory bowel disease (IBD) such as Crohn's disease and ulcerative colitis, irritable bowel syndrome (IBS), asthma, anaphylaxis, eosinophilic esophagitis, allergic rhinitis, and food allergies.

GluR6 (Glutamate lonotropic Receptor Kainate Type Subunit 2, also commonly referred to as GluK2) is an ionotropic glutamate receptor, and is classified a kainate receptor, which are known to involved in the regulation of synaptic transmission and plasticity in several brain regions. The structure of GluR6 is known (see, e.g., Nanao M H, et al. Structure of the kainate receptor subunit GluR6 agonist-binding domain complexed with domoic acid. Proc Nat/Acad Sci USA. 2005 Feb. 1; 102(5):1708-13). GluR6 is encoded by the gene GRIK2 (See, e.g., GeneCards® GC06P100962, OMIM 138244, and NCBI Gene ID 2898, among others). GRIK2 is very strongly-conserved in mammals, including humans, rhesus monkeys, chimpanzees, cats, dogs, horses, goats, sheep, cattle, pigs, mice, and rats, and shows strong conservation among vertebrates, including mammals, birds, reptiles, and fish.

Data herein show that agonists of the GluR6 receptor are effective at promoting mast cell, e.g. cutaneous mast cell, hyporesponsiveness, and therefor are expected to be able to treat diseases associated with mast cell activation, e.g., degranulation, such as, for example and without limitation: type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barre syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, a disease resulting from, or caused by, cutaneous mast cell activation, inflammatory bowel disease (IBD) such as Crohn's disease and ulcerative colitis, irritable bowel syndrome (IBS), asthma, anaphylaxis, eosinophilic esophagitis, allergic rhinitis, and food allergies.

Examples of agonists of the GluR6 receptor (GluR6 agonists) include agonists specific to the GluR6 receptor and non-specific glutamate receptor agonists that can act on the GluR6 receptor as well as one or more other glutamate (e.g., kainate) receptors. Proof of concept experiments for the ability of a GluR6 agonist to promote mast cell hyporesponsiveness were conducted, as outlined below, using SYM2081. SYM2081 may be synthesized by any useful method, such as that of Gu, Zi-Qiang, et al. (“Diastereoselective synthesis of (2S,4R)-4-methylglutamic acid (SYM 2081): a high affinity and selective ligand at the kainate subtype of glutamate receptors”, Bioorg. Med. Chem. Lett. (1995), 5(17), 1973-6). Other GluR6 agonists are expected to be useful for promoting mast cell hyporesponsiveness, including glutamate and kainic acid. Further 2,4-syn-functionalized (S)-glutamate analogs are described in Sagot E, et al. Chemo-enzymatic synthesis of a series of 2,4-syn-functionalized (S)-glutamate analogues: new insight into the structure-activity relation of ionotropic glutamate receptor subtypes 5, 6, and 7. J Med Chem. 2008 Jul. 24; 51(14):4093-103. Various patents describe suitable GluR6 agonists, including, without limitation: U.S. Pat. No. 5,731,348, EP 0 809 624 B1, and WO 2015/036618 A1, the disclosure of each of which is hereby incorporated herein by reference for their descriptions of GluR6 agonists. See, also, Donevan S D, et al. The methylglutamate, SYM 2081, is a potent and highly selective agonist at kainate receptors. J Pharmacol Exp Ther. 1998 May; 285(2):539-45; Zhou L M, et al. (2S,4R)-4-methylglutamic acid (SYM 2081): a selective, high-affinity ligand for kainate receptors. J Pharmacol Exp Ther. 1997 January; 280(1):422-7; and Jones K A, et al. Desensitization of kainate receptors by kainate, glutamate and diastereomers of 4-methylglutamate. Neuropharmacology. 1997 June; 36(6):853-63.

The GluR6 agonist may be a 4-substituted and/or N-substituted glutamine having the structure of Formula (I), as follows (carbons 2 and 4 are noted):

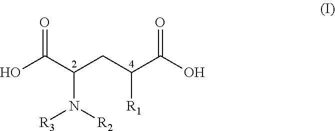

wherein:
   R₁ is C1-C3 alkyl, e.g. methyl, ethyl, n-propyl, isopropyl, or cyclopropyl, halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
   R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3,
or a pharmaceutically acceptable salt thereof (See, e.g., EP 0 809 624 B1). In examples, R₁ is methyl, and one of R₂ and R₃ is H and the other of R₂ and R₃ is H, methyl, —CHO, or COCH₃ in any stereo configuration, including any stereoisomeric mixtures thereof, such as enantiomeric mixtures. Combinations of GluR6 agonists may be utilized. In examples, the preceding GluR6 agonist of Formula (I) is a stereoisomer thereof, including (2S,4R), (2R,4S), (2S,4S), or (2R,4R) configurations, and mixtures of any combination of those stereoisomers at any useful % wt. for each.

In examples, the GluR6 agonist is 4-methylglutamic acid, including stereoisomers thereof, including (2S,4R)-4-methylglutamic acid (SYM 2081), (2R,4S)-4-methylglutamic acid, (2S,4S)-4-methylglutamic acid, or (2R,4R)-4-methylglutamic acid, or mixtures of any combination of those stereoisomers at any useful % wt. for each.

The GluR6 agonist may be glutamate, kainic acid, or domoic acid. Domoic acid is toxic but may be administered locally in small, non-toxic amounts, much in the way botulinum toxin is administered (see, e.g., Pulido O M. Domoic acid toxicologic pathology: a review. Mar Drugs. 2008 May 28; 6(2):180-219). That said, use of domoic acid may be less favored considering the efficacy of less-toxic drugs such as SYM2081.

Active ingredients, such as a GluR6 agonist, may be compounded, formulated, or otherwise manufactured into a suitable composition for use, such as a pharmaceutical dosage form, a topical dosage form, or drug product in which the GluR6 agonist is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, stabilizers, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. Additional non-limiting examples of useful excipients include disaccharides or sugar polyols (e.g., lactose, glucose, sorbitol, and maltitol).

Useful dosage forms include, without limitation: topical, parenteral, systemic, intravenous, intraperitoneal, intramuscular, intradermal, subdermal, mucosal, inhaled, nasal, or oral drug products. Topical products include, without limitation: ointments, creams, pastes, lotions, gels, drops, and transdermal devices (e.g., patches) and may be delivered, for example, via dermal, optic, or otic routes. In one non-limiting embodiment, the compound is a sterile solution comprising the active ingredient (drug, or compound), and a solvent, such as water, saline, lactated Ringer's solution, or phosphate-buffered saline (PBS), and may be delivered parenterally. Additional excipients, such a disaccharides or sugar polyols, polyethylene glycol, emulsifiers, salts, and buffers may be included in the solution. The drug product may be formulated as a topical product, and along with the active ingredient may include one or more of, for example and without limitation: skin permeation enhancers, rheology modifiers, emulsifiers, colorants, fragrance, buffers, salts, lipids, surfactants, among many others as are broadly-known in the pharmaceutical arts. Skin permeation enhancers, also known as chemical penetration enhancers (CPEs) or sorption promoters may be included and include, without limitation: terpenes, terpenoids, sulfoxides, laurocapram, pyrrolidones, fatty acids, fatty alcohols, alcohols containing glycols, urea, liposomes and surfactants (see, e.g., Raina N, et al. New Insights in Topical Drug Delivery for Skin Disorders: From a Nanotechnological Perspective. ACS Omega. 2023 May 19; 8(22):19145-19167).

Suitable dosage forms may include single-dose, or multiple-dose forms, such as, for topical use, an ointment tube or squeezable bottle containing the pharmaceutical composition comprising the GluR6 agonist. Additional dosage forms may include suitable liquid, solid, or semi-solid compositions. A liquid dosage form may be a solution, a suspension, or an emulsion. A solid dosage form may be a suppository, a capsule, a tablet, or a powder form. A semi-solid dosage form may be a gel, a foam, or a cream.

Pharmaceutical formulations adapted for administration include aqueous and non-aqueous sterile and non-sterile compositions which may contain, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials.

Therapeutic compositions typically must be sterile, though the topical compositions described herein need not be so. Therapeutic compositions typically must be stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. The proper fluidity of a solution can be maintained, for example, by the use of an emulsifier such as lecithin, by the maintenance of the required particle size in the case of dispersion, and by the use of surfactants and/or rheology modifiers. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweigh the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

An amount of any reagent or therapeutic agent, administered by any suitable route, effective to treat a patient is an amount capable of inducing mast cell hyporesponsiveness or treatment of a disease associated with mast cell activation. The therapeutically-effective amount of each therapeutic may range from 1 pg per dose to 10 g per dose from 1 μg to 1 mg per dose, including any amount there between, such as, without limitation, 1 ng, 1 μg, 1 mg, 10 mg, 100 mg, 1 g, or 10 g per dose; from 1 μg/h to 1 mg/h such as 10 μg/h, 100 μg/h, 200 μg/h, or 400 μg/h; or from 0.01 μg/kg/h to 10 μg/kg/hr, such as 1 μg/kg/hr, 5 μg/kg/hr, or 10 μg/kg/hr, where a dose may be an amount administered to a patient in a pharmaceutically-acceptable carrier.

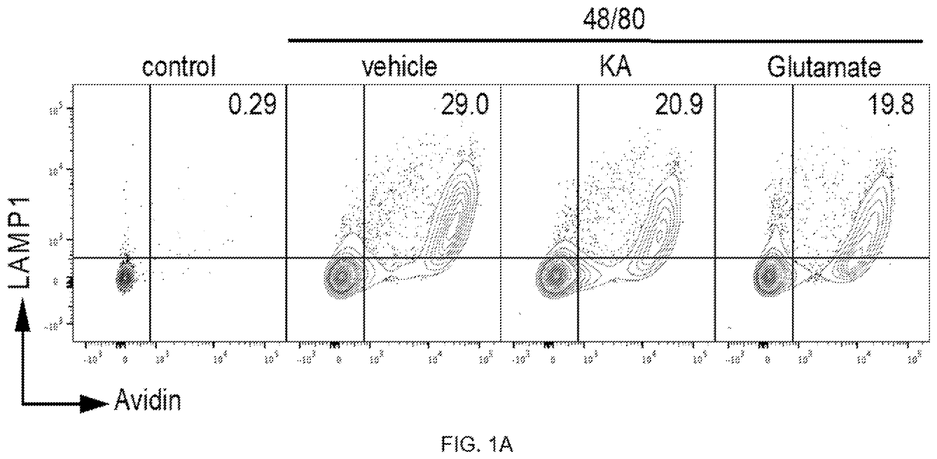

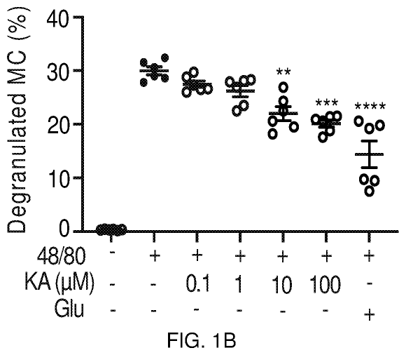

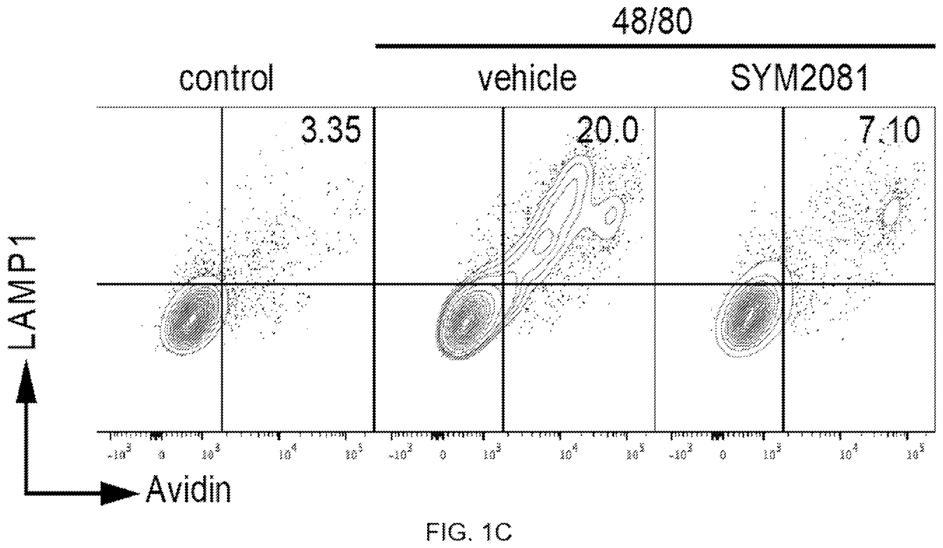

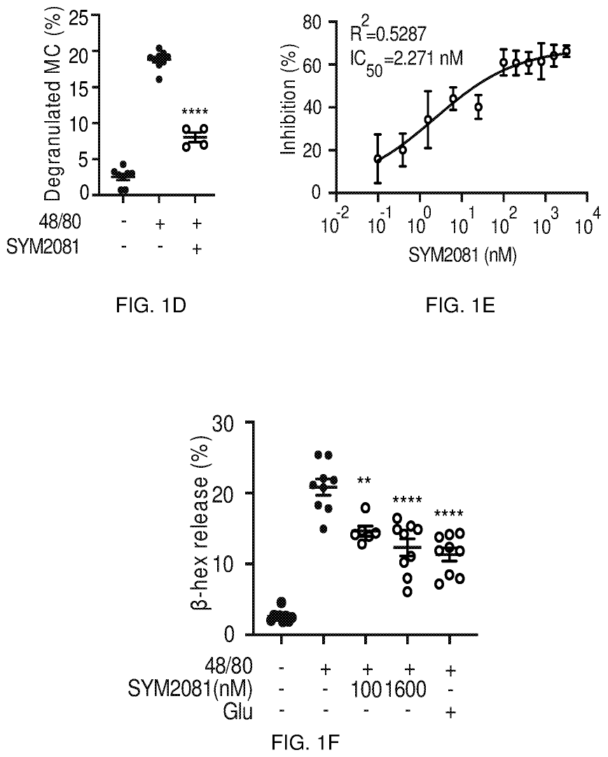

SYN2081 is shown below to be effective at a concentration ranging from at least 0.1 nM (nanomolar, or 100 picomolar) or greater (see, e.g., FIG. 1E). An upper concentration range limit may be a concentration at which undesirable side effects, e.g., toxicity, or no further beneficial effect is seen, e.g., at concentrations of greater than 10,000 nM (10 millimolar) as shown in FIG. 1E. As such an exemplary useful dosage range would be an amount of SYM2081 effective to produce a concentration in a patient ranging from 100 pM to 10 mM. Other GluR6 glutamate receptor agonists are expected to be effective in this ranges, e.g., greater than 100 pM, or ranging from 100 pM to 10 mM. In a further example, in vivo data showed that 1600 nM SYM2081 delivered intradermally, and a 5% SYM2081 topical composition were effective to reduce mast cell degranulation (e.g., to induce mast cell hyporesponsiveness). The amount of SYM2081 as an active agent administered to a patient may vary depending on the delivery route, and target tissue. Drug formulation and bioavailability may be gauged by empirical methods known to those of ordinary skill in the pharmaceutical arts.

Dosage regimens may be adjusted to provide an optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc. The composition may be proportionally reduced or increased as indicated by the exigencies of the therapeutic situation. In some instances, it may be especially advantageous to formulate compositions in dosage unit form for ease of administration and uniformity of dosage. The specification for the dosage unit forms may be dictated by and directly dependent on (a) the unique characteristics of the active compound and the particular therapeutic or prophylactic effect to be achieved, and (b) the limitations inherent in the art of compounding such an active compound for the treatment of individuals.

### Examples

Cutaneous mast cells underlie numerous skin inflammatory processes and have anatomical and functional associations with sensory afferent neurons. The ablation of a subset of sensory nonpeptidergic neurons expressing MrgprD increased the expression of a mast cell gene module, including the activating receptor, Mrgprb2, leading to increased mast cell reactivity to Mrgprb2 ligands, such as compound 48/80, and cutaneous inflammation in several disease models, such as irritant dermatitis, rosacea, and contact hypersensitivity. MrgprD-expressing neurons released glutamate to inhibit mast cell reactivity. Reducing glutamate release from MrgprD-expressing neurons in the periphery resulted in a hyperresponsive state of mast cells. The antagonism of glutamate receptors on cultured peritoneal cavity mast cells (PCMCs) or dermal mast cells enhanced MC degranulation and phenocopied the genetic state in the absence of MrgprD-expressing neurons. We then identified the kainate receptor GluR6/KA2 as the glutamate receptor subtype expressed on dermal MCs. Two non-specific agonists of GluR6, glutamate, and kainic acid, and the highly selective GluR6 agonist SYM2081 suppressed the degranulation of PCMC in response to MrgprB2 but not FcsRI crosslinking in a dose-dependent manner in vitro. In vivo, intradermal delivery of SYM2081 suppressed compound 48/80 induced-dermal MC activation, and attenuated croton oil-induced irritant dermatitis, but not IgE-dependent passive cutaneous anaphylaxis. Together, these data demonstrated that glutamate acting via the GluR6/KA2 receptor promotes mast cell hyporesponsiveness during homeostasis and suggests that selective agonism of this pathway has the potential to reduce mast cell activation in vivo which would be of significant utility in the treatment of diseases in which mast cells represent an important component of the pathogenic pathway.

At present there are no highly effective mechanisms to suppress the activation of cutaneous mast cells. By agonizing the GluR6 glutamate receptor on mast cells we can reprogram mast cells into a state of low responsiveness. The use of GluR agonists to target immune cells in novel as is targeting cells in the periphery. GluR agonism has been attempted for the treatment of CNS diseases but not in the periphery.

Synthesis of SYM2081. Enantiometrically pure (2S,4R)-4-methylglutamic acid may be diastereoselectively synthesized as shown in Gu, Zi-Qiang, et al., Bioorg. Med. Chem. Lett. (1995), 5(17), 1973-6.

In vitro data to support suppression of mast cell activation SYM2081 agonism of GluK2. Primary mast cell cultures derived from murine peritoneal mast cells were incubated for 48 hours with kainic acid (0.1, 1.0, 10 and 100 uM), glutamate (6 mM) or vehicle control. Cultures were then exposed to the synthetic MrgprB2 ligand, compound 48/80, at 20 ug/ml for 20 minutes. The extent of mast cell degranulation was determined based on surface expression of Lamp1 protein and avidin binding using flow cytometry gated on CD117+ FcεR+ mast cells. Representative flow plots from unstimulated cells (control) and cells stimulated with compound 48/80 are shown (FIG. 1A). Quantification of mast cell degranulation across multiple samples is shown (FIG. 1B) demonstrating that incubation with glutamate which binds to all glutamate receptors and kainic acid which binds to kainic glutamate receptors both suppress mast cell activation. A similar set of experiments were performed using mast cells incubated with the selective GluK2 agonist, SYM2081 at 1600 nM concentration (FIGS. 1C and 1D). A dose response with various concentrations of SYM2081 found that this compound suppressed mast cells with an IC₅₀ of 2.271 nM (FIG. 1E). Finally, we confirmed suppression of mast cell degranulation by SYM2081 at 100 nM and 1600 nM based on release of the enzyme β-hexosaminidase which is contained within mast cell granules (FIG. 1F). Consistent with our flow cytometry-based degranulation assay, release of β-hexosaminidase was decreased in mast cells preincubated with SYM2081. * p<0.05; **p<0.01; ***p<0.0001; ****p<0.00001. Each symbol represents data from a single well of mast cells.

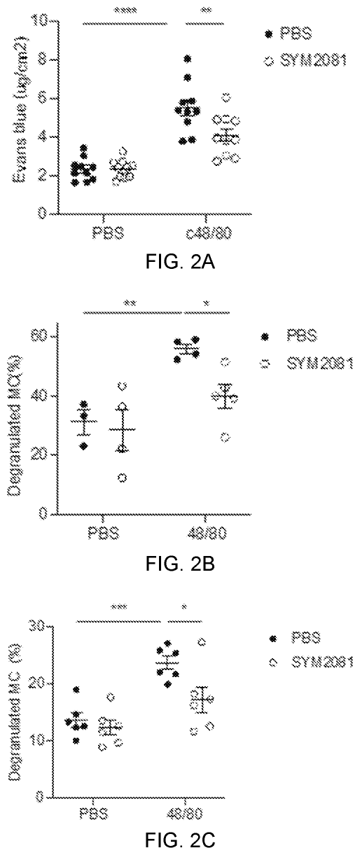

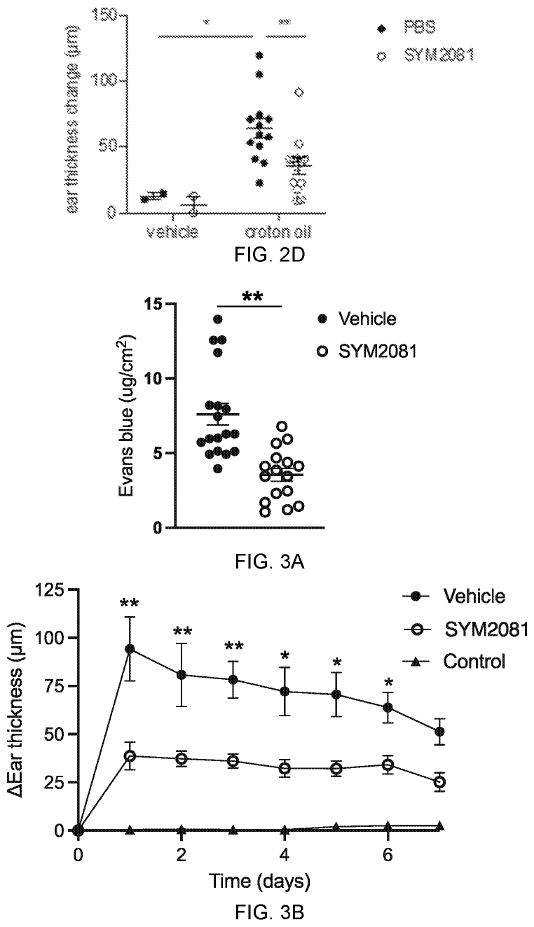

In vivo data to support suppression of mast cell activation with SYM2081 agonism of GluK2. Cohorts of 7-9-week-old female C57BL/6 mice were given 20 ul intradermal flank injections of 1600 nM SYM2081 or control PBS twice a day for 2 days. Evans Blue dye was introduced by intravenous injection to allow for quantification of edema followed by intradermal injection of control PBS or compound 48/80 (20 ul 10 ug/ml). The amount of Evans blue dye released into the skin which quantifies edema and is an indirect measure of mast cell degranulation was measured 20 minutes later (FIG. 2A). Mice treated with SYM2081 showed a significant reduction in edema indicating reduced mast degranulation in response to compound 48/80. The percentage of degranulated mast cells was also quantified by direct microscopic visualization of skin samples stained with toluidine blue (FIG. 2B). Whole human skin explants were cultured in control PBS or 1600 nM SYM2081 for 48 hours. Sample were exposed to compound 48/80 (240 ug/ml) for 20 minutes followed by direct microscopic visualization of skin samples stained with toluidine blue (FIG. 2C). Skin explants exposed to SYM2081 showed a reduced percentage of degranulated mast cells following exposure to compound 48/80 demonstrating reduced activation. Finally, C57BL/6 mice were treated with intradermal injection of control PBS or SYM2081 for 2 days as above. Mice were then challenged by application of 0.5% croton oil, an irritant, on 1 ear and the degree of ear swelling which is reflective of the extent of irritant dermatitis was measured (FIG. 2D). Mice pretreated with SYM2081 showed reduced ear swelling indicated a reduction in the degree of irritant dermatitis in response to croton oil. * p<0.05; **p<0.01; ***p<0.0001; ****p<0.00001. Each symbol represents data from an individual animal.

In vivo data to support suppression of mast cell activation with topical SYM2081. Cohorts of 7-9-week-old female C57BL/6 mice were treated with 5.0% SYM2081 formulated into a light base or vehicle alone on the flank twice daily for 2 days. Evans Blue dye was introduced by intravenous injection to allow for quantification of edema followed by intradermal injection of control PBS or compound 48/80 (20 ul 10 ug/ml). The amount of Evans blue dye released into the skin which quantifies edema and is an indirect measure of mast cell degranulation was measured 20 minutes later (FIG. 3A). Mice treated with SYM2081 showed a significant reduction in edema indicating reduced mast degranulation in response to compound 48/80. In a model of contact hypersensitivity, a separate cohort were treated with topical SYM2081 or vehicle on the shaved abdomen twice daily for 2 days. Mice were then sensitized with 0.5% DNFB on the abdomen. Five days later mice were challenged with 0.25% DNFB on one ear and increased ear thickness as a measure of inflammation was measured for the following 7 days (FIG. 3B). Mice treated with SYM2081 showed reduced inflammation in response to DNFB compared with vehicle treated animals. * p<0.05; **p<0.01. Each symbol represents data from an individual animal (FIG. 3A) or mean+/−SEM of 10 animals per group (FIG. 3B).

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of treating a disease associated with activation of mast cells in a patient, such as a vertebrate, mammalian, or human patient, comprising administering to the patient an amount of a GluR6 glutamate receptor agonist effective to reduce responsiveness of a mast cell in the patient.

2. The method of claim 1, comprising inducing hyporesponsiveness in the mast cells.

3. The method of claim 1, wherein the mast cells are cutaneous mast cells.

4. The method of claim 1, wherein the disease is one or more of type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barré syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, a disease resulting from, or caused by, cutaneous mast cell activation, inflammatory bowel disease (IBD) such as Crohn's disease and ulcerative colitis, irritable bowel syndrome (IBS), asthma, anaphylaxis, eosinophilic esophagitis, allergic rhinitis, and food allergies.

5. The method of claim 1, wherein the disease is one of atopic dermatitis, psoriasis, rosacea, eczema, contact dermatitis, mastocytosis, skin inflammation urticaria, urticarial diseases in a patient.

6. The method of claim 1, wherein the GluR6 glutamate receptor agonist is administered topically to the patient.

7. The method of claim 1, wherein the GluR6 glutamate receptor agonist comprises 2-Amino-4-methylpentanedioic acid, or a pharmaceutically-acceptable salt thereof, (2S,4R)-2-Amino-4-methylpentanedioic acid, or a pharmaceutically acceptable salt thereof, glutamate, kainic acid, or domoic acid.

8. The method of claim 1, wherein the GluR6 glutamate receptor agonist comprises a compound having the structure of Formula (I), as follows: 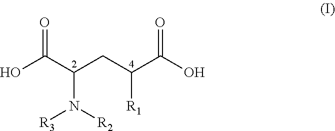
wherein:
R₁ is C1-C3 alkyl (e.g. methyl, ethyl, n-propyl, isopropyl, or cyclopropyl), halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3,
or a pharmaceutically acceptable salt thereof.

9. The method of claim 8, wherein R₁ is methyl, and one of R₂ and R₃ is H and the other of R₂ and R₃ is H, methyl, —CHO, or COCH₃.

10. The method of claim 1, wherein the GluR6 glutamate receptor agonist is administered to a concentration ranging from 100 pM (picomolar) to 10 mM (millimolar).

11. A method of suppressing activation of mast cells, comprising contacting the mast cell with a GluR6 glutamate receptor agonist, in an amount effective to reduce responsiveness of the mast cell.

12. The method of claim 11, comprising inducing hyporesponsiveness in the mast cells.

13. The method of claim 11, wherein the mast cells are cutaneous mast cells.

14. The method of claim 11, for treatment of a disease associated with mast cell activation in a patient, such as a vertebrate, mammalian, or human patient, comprising administering to the patient an amount of a GluR6 glutamate receptor agonist effective to reduce responsiveness of a mast cell in the patient.

15. The method of claim 14, wherein the disease is one or more of type I hypersensitivity reactions (e.g., allergies), mastocytosis, pseudo-allergy, mast cell activation syndrome, atopic dermatitis, psoriasis, rosacea, eczema, contact dermatitis, urticaria, urticarial diseases, Type I diabetes, Guillain-Barré syndrome, bullous pemphigoid, wound healing, exuberant arthropod reactions, Sjogren syndrome, atherosclerosis, coronary inflammation, cardiac ischemia, chronic idiopathic urticaria, Ehlers-Danlos Syndromes, skin inflammation, cancer, a disease resulting from, or caused by, cutaneous mast cell activation, inflammatory bowel disease (IBD) such as Crohn's disease and ulcerative colitis, irritable bowel syndrome (IBS), asthma, anaphylaxis, eosinophilic esophagitis, allergic rhinitis, and food allergies.

16. The method of claim 11, wherein the GluR6 glutamate receptor agonist is administered topically to the patient.

17. The method of claim 11, wherein the GluR6 glutamate receptor agonist comprises:
a compound having the structure of Formula (I), as follows: 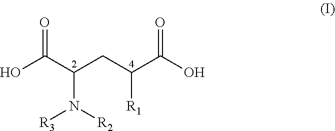
wherein:
R₁ is C1-C3 alkyl (e.g. methyl, ethyl, n-propyl, isopropyl, or cyclopropyl), halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3,
or a pharmaceutically acceptable salt thereof;
2-Amino-4-methylpentanedioic acid, or a pharmaceutically-acceptable salt thereof;
(2S,4R)-2-Amino-4-methylpentanedioic acid, or a pharmaceutically acceptable salt thereof;
glutamate;
kainic acid; or
domoic acid.

18. The method of claim 11, wherein the GluR6 glutamate receptor agonist is contacted with the mast cells at a concentration ranging from 100 pM (picomolar) to 10 mM (millimolar).

19. A topical drug composition, comprising GluR6 glutamate receptor agonist and a pharmaceutically-acceptable excipient.

20. The composition of claim 19, comprising: a compound having the structure of Formula (I), as follows: 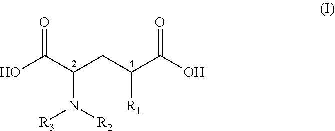
wherein:
R₁ is C1-C3 alkyl (e.g. methyl, ethyl, n-propyl, isopropyl, or cyclopropyl), halogen, —CH₂—OH, —CH₂—O—CH₂-Ph (Ph is phenyl), —CH₂—C(O)OH(—C(O)OH is carboxyl), —CH₂—C(O)O—CH₃, —CH₂—CH₂—C(O)OH, —CH₂—CH₂—C(O)O—CH₃, —CH₂—CH₂—C(O)—NH₂, —CH₂—CH₂—C(O)—NH—CH₃, —CH₂—CH₂—C(O)—NH—CH₂—CH₃, —CH₂—CH₂—C(O)—NH—CH₂—CH₂—CH₃, or —CH₂—CH₂—C(O)—NH—CH₂-Ph, and
R₂ and R₃ are independently H, C1-C6-alkyl, C3-C4-alkenyl, C3-C5-cycloalkyl, C1-C6-alkyl-CO—, C1-C6-alkyl-OCO—, C1-C6-alkyl-NHCO—, CHO—, or C3-C6-alkynyl, and R₂ and R₃ taken together can be —CH₂(CH₂)ₚCH₂—, where p is 0, 1, 2 or 3, or a pharmaceutically acceptable salt thereof, such as 2-Amino-4-methylpentanedioic acid, or a pharmaceutically-acceptable salt thereof, or (2S,4R)-2-Amino-4-methylpentanedioic acid, or a pharmaceutically acceptable salt thereof,
in a concentration (e.g., % wt., or any useful measure of concentration or amount of an active ingredient in a composition) for delivery of an amount of the GluR6 glutamate receptor agonist effective to treat a disease associated with activation of mast cells in a patient.
